(19) United States
(12) Patent Application Publication
(10) Pub. No.: US 2017/0290762 A1
(43) Pub. Date: Oct. 12, 2017

(54) Method and Composition for Dag Mitigation on Hair

(71) Applicant: Empire Technology Development LLC, Wilmington, DE (US)

(72) Inventor: Georgius Abidal Adam, Edensor Park (AU)

(21) Appl. No.: 15/094,436

(22) Filed: Apr. 8, 2016

(51) Int. Cl. A61K 8/89; A61K 8/49

(52) U.S. Cl. CPC A61K 8/89; A61K 8/4973

(57) ABSTRACT

Technologies are described for a method and constituents for mitigating dag on hair. The method comprises applying a hydrophobic silicon containing constituent and a reactive constituent onto the hair. The reactive constituent enables a reaction with the hair that binds the hydrophobic silicon constituent to the hair and the hydrophobic silicon constituent imparts its hydrophobicity to the hair to mitigate dag on the hair.

### TECHNICAL FIELD

The present disclosure is directed generally towards a method and composition for dag mitigation, and more specifically towards mitigating dag on the hair of animals, especially livestock.

### BACKGROUND

Dag, Daglock, or daggle-lock is a lumpy, dirty, or clotted hair mass that has accumulated on the hair of livestock, such as cattle or sheep, and other animals. For example, dag may be a dangling or matted lock of fur, hair, or wool and may comprise feces or urine. For this disclosure, a dag is considered to be any foreign matter that clings to the hair, wool or other mammalian hair of an animal.

Dag often forms with water, urine, and defecation, which may become a part of a concrete-like composite. The formation of dag may lead to an enhanced probability of disease as large amounts of bacteria may be introduced into the dag. Dag formation on cattle, and problems associated therewith, may be prevalent in many places. For example, cattle are often exported in large ships. The dags may add considerable weight and should be removed from the cattle before boarding to prevent excess shipping weight, disease, and for passing inspection.

Dags adhered to the hair of livestock may also represent a significant health problem for the beef processing industry. This may be especially true during seasonal periods of rain. Processing of “daggy” cattle at abattoirs may increase the risk that meat reaching the consumer is contaminated with pathogenic microorganisms.

Current methods for dag removal may induce stress in live animals at feedlots. This may have a detrimental effect on the quality of meat reaching the consumer, and additionally may pose health and safety hazards for workers. While cleaning and removal of the dags may be performed at the abattoir before or following slaughter, high microbial loads may be present in dags which may threaten food safety protocols.

Unless otherwise indicated herein, the materials described in this section are not prior art to the claims in this application and are not admitted to be prior art by inclusion in this section.

### SUMMARY

Technologies are generally described for a method and constituents for mitigating dag on hair. Mitigating dag may ease the removal of the dag or mitigate its formation on hair. Mitigating the formation of dag on hair may reduce, or even eliminate, the need to remove the dag. A method and composition for mitigating dag formation on hair is presently disclosed herein. The method comprises applying one or more constituents or compounds onto hair, wherein the applied constituents comprise a hydrophobic silica containing constituent and a reactive constituent. The application of the constituents onto the hair enables a reaction of the reactive constituent with the hair and transfer of the hydrophobicity of the hydrophobic silicon containing constituent to the hair.

The foregoing summary is illustrative only and is not intended to be in any way limiting. In addition to the illustrative aspects, embodiments, and features described above, further aspects, embodiments, and features will become apparent by reference to the examples and drawings and the following detailed description.

### BRIEF DESCRIPTION OF THE FIGS.

The foregoing and other features of this disclosure will become more fully apparent from the following description and appended claims, taken in conjunction with the accompanying drawings and examples. Understanding that these drawings depict only several embodiments in accordance with the disclosure and are, therefore, not to be considered limiting of its scope, the disclosure will be described with additional specificity and detail through use of the accompanying drawings, in which:

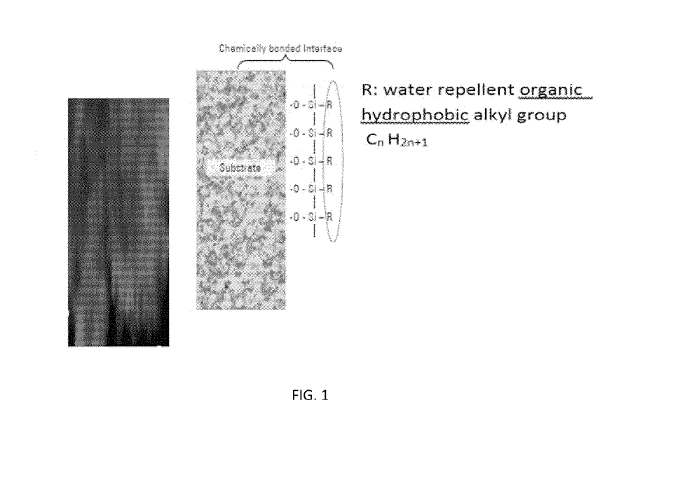

FIG. 1 illustrates a surface of an animal on the left plate and an underlying chemically bonded interface of a hydrophobic silicon containing constituent on the right plate;

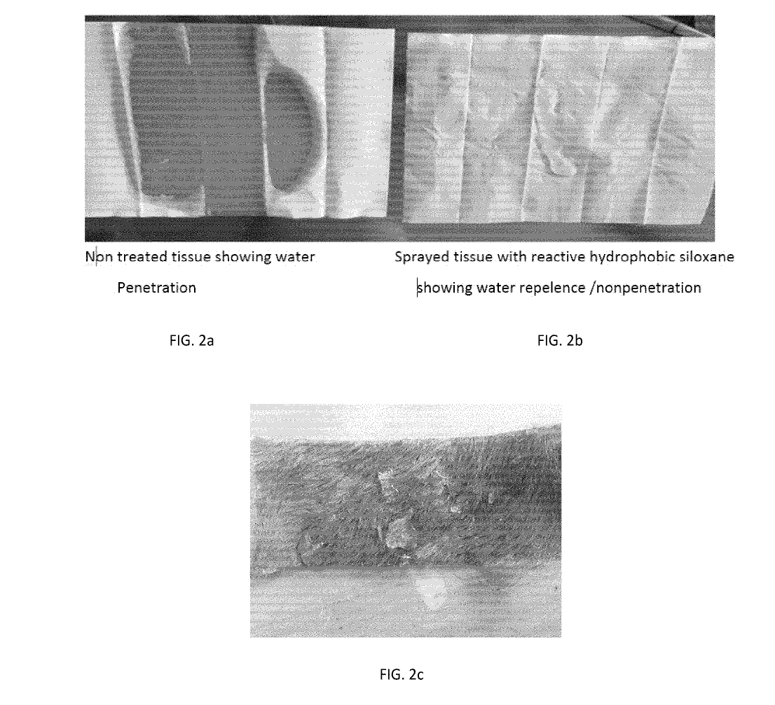

FIGS. 2a-2c illustrate water repellency or hydrophobic characteristics of applied silicon constituents;

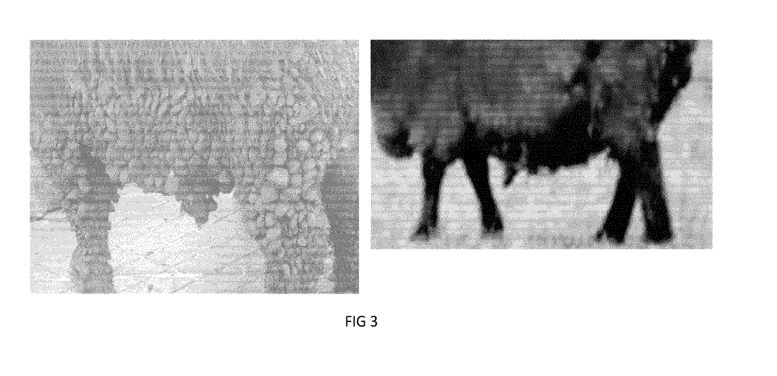

FIG. 3 illustrates photos of a livestock before and after treatment;

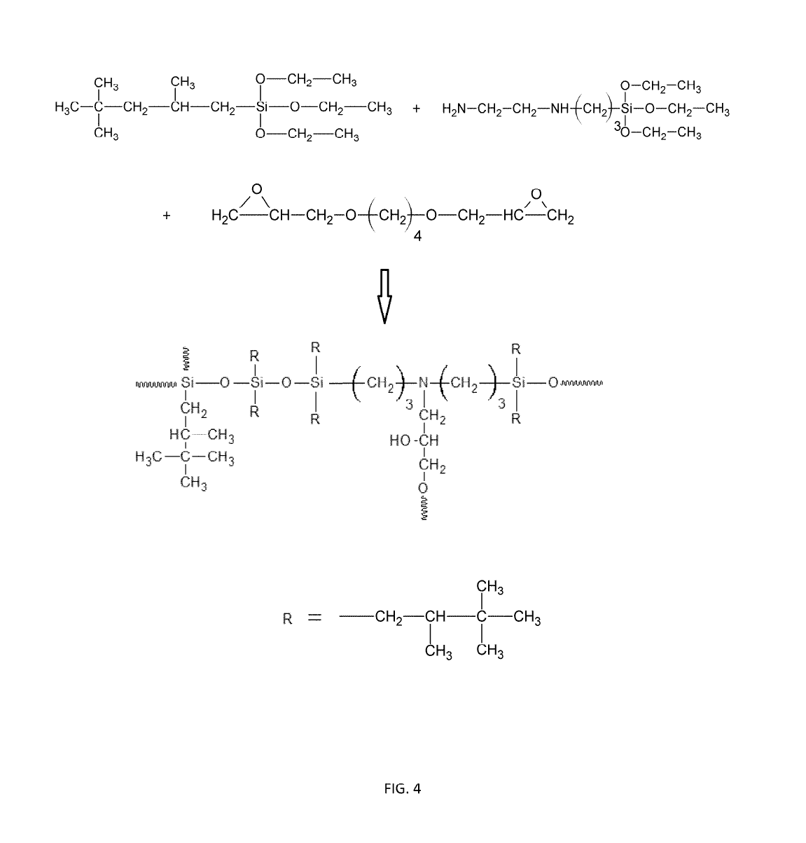

FIG. 4 illustrates a reaction scheme; and

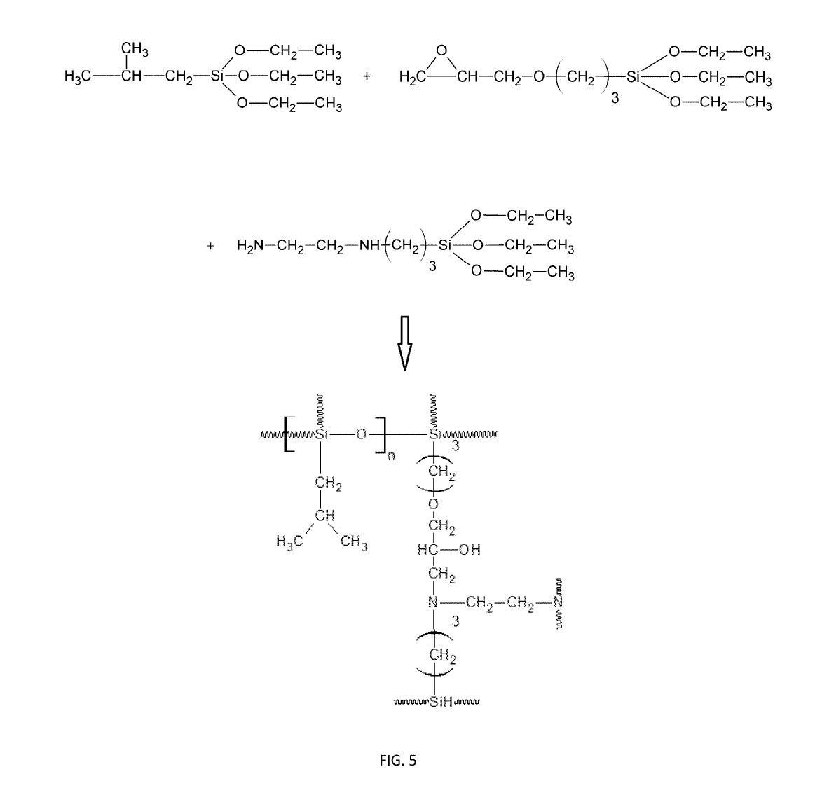

FIG. 5 illustrates another reaction scheme.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the FIGS., can be arranged, substituted, combined, separated, and designed in a wide variety of different configurations, all of which are explicitly contemplated herein.

This disclosure is generally drawn, inter alia, to methods and constituents for mitigating dag formation on hair. Briefly stated, technologies are generally described for a method and constituents, or compounds, for reducing, mitigating, substantially eliminating the formation of dag on hair or easing the removal of dag from hair. In an illustrative method, a hydrophobic silicon containing constituent and a reactive constituent are applied to hair. The hydrophobic silicon containing constituent has at least one functional group selected from the group consisting of an epoxy group, a reactive amino group, a reactive cyanoacrylate group, a reactive isocyanate group, oxime, and combinations thereof. The applied reactive constituent enables a reaction with the hair that binds the applied hydrophobic silicon to the hair and the applied hydrophobic silicon containing constituent enables a transfer of its hydrophobicity to the hair to mitigate dag on the hair.

The term hair is used broadly herein to mean any form of mammalian hair such as wool or any of the fine threadlike strands growing from the skin. The term hide is used broadly herein to mean the skin from which the hair is growing. For example, the method and constituents of the present disclosure may reduce or even eliminate a need to remove dag from the hair of cattle. It will be appreciated that by reducing or even eliminating dag on the hair of cattle, this disclosure helps keep dag away from the hide of the cattle or the formed dags are weak and can easily be removed.

As previously explained, for purposes of this disclosure, a dag is considered to be any foreign matter that clings to the hair, wool or other covering of an animal.

The current disclosure may provide a non-stress-inducing method of mitigating the formation of dags. The mitigation of the formation of dags may mitigate the contamination of the meat. The reduction, mitigation, or elimination of the formation of dags may, in turn, aid the slaughter house in meeting regulations that may require that the cattle be deemed “clean” prior to slaughter. In addition, post-slaughter de-lagging methods may not be a viable option. This may be especially true in meeting Australian regulations.

The invention is based on using silicon based hydrophobic constituents, reactive silicon based hydrophobic agents, and/or water repellent ingredients that may become reactive with keratin, melanin, or other hair components. A reaction of the constituents with hair, for example cattle hair, may cause a hydrophobic constituent to become chemically bonded with hair. The hydrophobic constituent may became part of the cattle dressing and keep the cattle dry and clean. The method may be tailored to be persistent in keeping the hair substantially dry for several months or until the hair of the cattle is renewed or the cattle is slaughtered.

The reaction of the constituents with the hair may be a mild non-exothermic reaction, based on a reaction of room temperature vulcanization (RTV), silicon reactive groups, epoxy groups, urethane. A reaction mechanism that may take place may be based on changing the structure of cysteine and hydrogen bonding groups of cysteine: COOH, NH2, NH, C═O, SH, CH₂═CH— to cysteine or melanin bonded to the hydrophobic constituent(s), agent(s), or water repellent(s).

Several types of reactive constituents may be applied for bonding a hydrophobic constituent of this disclosure to hair. For example, some reactive constituents may be commercially available and used as cross-linking agents for silicon rubber, epoxy resins, or photocopying, as is known in the art. The method of mitigating dag on hair of the present disclosure may be non-stressing to cattle and easy to apply, by spraying for example. The method may also provide for the application of nontoxic active ingredients or constituents, ambient temperature curing, moisture catalytic curing, cationic catalytic curing, and visible light photo curing.

The present disclosure may provide a new method of mitigating or preventing dags to be formed on the hair of the live cattle as a non-stress-inducing method. The method may provide for an application to live cattle before the wet winter months, for example. The presently disclosed method may not involve activities and factors that are known to be stressful to cattle such as noisy environment, human handling, electric prodding, washing, dipping, brushing, and shearing.

The selective ingredients, constituents, or compounds presently disclosed may protect the hair of cattle from wetting. Since dags may need wet hair to form, the mitigation of wetting of the hair may then in turn mitigate the formation of dags on the hair. This may be especially true during the rainy or winter months. The presently disclosed method and constituents may protect or inhibit wetting of the hair for several months.

Direct application or spraying of known hydrophobic agents and water repellents onto cattle hair to prevent dag accumulation may not be successful because cattle hair is hydrophilic. This may be due to the hydrogen bonding present in the amino acids of keratin, a component of hair. Cysteine is hydrophilic and is a basic binding constituent of keratin fibers. Therefore, hair is hydrophilic and wets easily when in contact with water. As is known in the art, hydrophilic and hydrophobic substances repel one another. Consequently, a direct application of known hydrophobic agents may not adhere to hair or may be washed out easily, and therefore may not mitigate dags.

The present disclosure provides a method of using a hydrophobic constituent(s), reactive hydrophobic agent(s) that are reactive, or enabled to become reactive, with keratin or Cysteine, for transferring the cattle hair to a protective substantially non-wettable hydrophobic composition that prevents or mitigates the accumulation of dags on hair. Dags may have a composition that is mostly hydrophilic, due to the presence of partially digested cellulose and sugar residues, which may also contribute to the mitigation of dag on treated hair, treated by the presently disclosed method.

In at least one embodiment of the present disclosure, a method of transforming the hair to be hydrophobic by reacting hydrophobic, or super hydrophobic, reactive silicon based active agent(s), or constituent(s), with keratin and/or cysteine, the essential component of hair, is provided. Keratin is a protein formed by the combination of 18 amino acids, among which cysteine being rich in sulphur plus other reactive functional groups that, that may play an important role in the cohesion of hydrophobic constituents to the hair. Cysteine represents the major active component of keratin, which may be used for changing the properties of hair from hydrophilic to hydrophobic. Coloring pigments in hair, such as melanin, may also have reactive functional groups which may also be effective in transforming hair from hydrophilic to hydrophobic.

Disclosed herein is a method of mitigating dag formation on hair. The method involves applying, for example spraying, constituents onto the hair. One or more of the constituents may be hydrophobic and may function as a water proofing agent or water repellant. Constituents of the present disclosure applied to the hair may have a hydrophobic silicon containing constituent and a reactive constituent. The applying of the constituents may enable an in-situ reaction of the reactive constituent with the hair. The reaction may cause the hydrophobic silicon containing constituent to be bonded with, or otherwise held to, the hair and thereby transfer its hydrophobicity to the hair.

A hydrophobic silicon containing constituent and a reactive constituent may be applied to the hair separately. For example, one of the hydrophobic silicon containing constituent that consist at least one reactive functional group may be applied to the hair or the silicon containing constituent and the reactive constituent may be applied to the hair as one composition usually mixed before application.

A hydrophobic silicon containing constituent and the reactive constituent may be applied to the hair simultaneously or separately. For example, the hydrophobic silicon containing constituent and the reactive constituent may be mixed together and applied to the hair in a single application. In at least one illustrative example, and the hydrophobic silicon containing constituent and the reactive constituent are parts of a single compound that may be applied in a single application.

One or more constituents may be in liquid form and the step of applying the constituents to the hair may include spraying, rinsing, dispersing, or applying by means which are known by persons having ordinary skill in the art for applying a liquid to hair. One or more constituents may be in solid or powder form, the solid reactive part may be soluble in the silicon liquid constituent and the step of applying the constituents to the hair spraying, sprinkling, or applying by means which are known by persons having ordinary skill in the art for applying liquids to hair.

Additional materials may also be included in the composition before being applied to the hair. For example, one or more hardeners, catalysts, solvents reactive diluents, bonding, crosslinking agents or other materials may be applied to the hair which may enhance, speed up, or drive a bonding reaction which may hold the hydrophobic silicon containing constituent to the hair.

In describing more fully this disclosure, we make reference to the accompanying drawings and following examples in which illustrative embodiments of the present disclosure are shown. This disclosure may, however, be embodied in a variety of different forms and should not be construed as so limited.

FIG. 1 illustrates a surface of an animal on the left plate and an underlying chemically bonded interface of a hydrophobic silicon containing constituent on the right plate. The left plate depicts the surface of the animal. Shown in the right plate is an interface of this disclosure formed by applying a composition having a hydrophobic silicon containing constituent and a reactive constituent to hair. As depicted in the right plate and explained below, the applied reactive constituent enables a reaction with the hair that binds the applied hydrophobic silicon containing constituent to the hair and the applied hydrophobic silicon containing constituent enables a transfer of its hydrophobicity to the hair to mitigate the formation of dag on the hair.

For example, the substrate may be livestock hair and the hydrophobic constituents may contain silicon active ingredient and have at least one hydrophobic alkyl group extending from the substrate. The hydrophobic alkyl group may comprise CₙH₂ₙ₊₁, wherein n=1 through 12. The hydrophobic alkyl group may be linear or branched. The reactive constituent may have alkoxy, epoxy group, amino group, oxime group, SH, CH═CH₂ or any other reactive functional group and may function to bind the hydrophobic constituents to the substrate, as is illustrated in FIG. 1.

FIGS. 2a-2c illustrate water repellency or hydrophobic characteristics of applied constituents. FIG. 2a, left, shows untreated tissue paper that has been sprayed with water. As shown in FIG. 2a, the untreated tissue paper is hydrophilic and has absorbed the water to an extent where portions of the tissue paper were disintegrated or were otherwise broken apart. FIG. 2b, depicted on the right, shows tissue paper that has been treated by the method of the present disclosure and then sprayed with water. More specifically, the tissue paper in the second illustration was treated by spraying it with a reactive hydrophobic siloxane in accordance with the presently disclosed method. As shown in the second illustration, the treated tissue is made hydrophobic due to the applied constituents of this disclosure and has repelled the sprayed on water to the extent that the water has pooled on its surface. FIG. 2c, depicted on the bottom, shows hair that has been treated by the method of the present disclosure and then sprayed with water. As shown in FIG. 2c, the treated hair is made hydrophobic due to the applied constituents of this disclosure and has repelled the sprayed on water to the extent that the water has pooled on the hair.

A comparison of FIG. 2a with FIGS. 2b and 2c clearly shows that the application of a hydrophobic silicon containing constituent and a reactive constituent, according to the present disclosure, enables a reaction with the substrate and transfers the hydrophobicity of the hydrophobic silicon containing constituent to the substrate to which it is applied.

FIG. 3 illustrates photos of a livestock before and after treatment for mitigating formation of dag on hair the livestock. The first illustration in FIG. 3, depicted on the left, shows an example of untreated livestock that has been raised in a typical farm environment. As shown in the first illustration, the untreated hair has accumulated a substantial amount of dag. The second illustration in FIG. 3, depicted on the right, shows an example of a livestock that has been treated with the constituents of the present disclosure and raised in a typical farm environment. The treated livestock in the second illustration was treated by spraying it with a reactive hydrophobic siloxane according to the presently disclosed method. As shown in the second illustration, the treated livestock hair is hydrophobic and the treatment of the hair has substantially mitigated, and in some cases may even eliminate, the formation of dag.

A comparison of the first and second illustrations of FIG. 3 clearly shows that the application of a hydrophobic silicon containing constituent and a reactive constituent, according to the present disclosure, onto hair of livestock enables a reaction with the hair, transfers the hydrophobicity of the hydrophobic silicon containing constituent to the hair.

Presently disclosed is a method of mitigating dag formation on hair. The method comprises applying a hydrophobic silicon containing constituent onto hair. The hydrophobic silicon containing constituent may have at least one functional group selected from the groups consisting of an epoxy group, a reactive amino group, a reactive cyanoacrylate group, a reactive isocyanate group, an oxime group, and combinations thereof. The method also comprises applying a reactive constituent onto the hair. The applying of the hydrophobic silicon containing constituent and the reactive constituent onto the hair enables a reaction with the hair and a transfer of the hydrophobicity of the hydrophobic silicon containing constituent to the hair. For example, the reactive constituent may react with keratin or cysteine in the hair and hold the hydrophobic silicon containing constituent to the hair.

The applying of the hydrophobic silicon containing constituent and the reactive constituent may be performed simultaneously. The reactive constituent may be applied simultaneously with the hydrophobic silicon containing constituent, in a single application. For example, the reactive constituent may be mixed with the hydrophobic silicon containing constituent and immediately applied. Alternatively, a single compound may have both the hydrophobic silicon containing constituent and the reactive constituent pre-mixed and may be applied in a single application.

Regardless whether a compound or mixture having both the hydrophobic silicon containing constituent and the reactive constituent is applied in a single application or the hydrophobic silicon containing reactive functional groups constituent upon the application of both compositions to the hair, they may react with the hair and bond or hold the hydrophobic silicon containing constituent to the hair. The hydrophobicity of the hydrophobic silicon containing constituent may thus be transferred to the hair.

More specifically, in each case, the applied reactive constituent enables a reaction with the hair that binds the applied hydrophobic silicon to the hair and the applied hydrophobic silicon containing constituent enables a transfer of its hydrophobicity to the hair to mitigate the formation of dag on the hair.

In at least one illustrative example, the applying of the hydrophobic silicon containing constituent is performed simultaneously with the applying a reactive constituent onto the hair, in a single application. For example, the hydrophobic silicon containing constituent and the reactive constituent may be comprised in a single compound. The one or more of the compounds may include those listed in Table 1, below. These compounds may have a reactive hydrophobic silicon constituent which may be applied to hair in a single application to mitigate dag formation.

TABLE 1

| | |
|---|---|
| 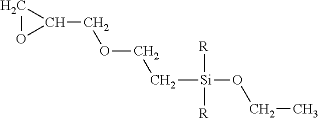 | reactive hydrophobic silicon containing constituent, wherein R = CₙH₂ₙ₊₁, n = 1-12, linear or branched |
| 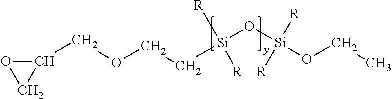 | reactive hydrophobic siloxane containing constituent, wherein R = CₙH₂ₙ₊₁, n = 1-12, linear or branched, and y = 1-4 |
| 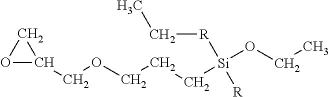 | reactive hydrophobic silicon containing constituent, wherein R = CₙH₂ₙ₊₁, n = 1-12, linear or branched |

The method may comprise an application of a reactive hydrophobic silicon containing constituent, or compound, to the hair. The hydrophobic silicon containing constituent, or reactive compound, may comprise siloxane. A reactive hydrophobic silicon may contain two or more reactive groups, for example epoxy and ethoxy groups.

A photocatalyst may also be applied onto the hair. The photocatalyst may be applied simultaneously with the reactive hydrophobic silicon containing constituent. Upon exposing the hair to light, a photocatalytic reaction of the reactive hydrophobic silicon containing constituent with the hair may take place.

A composition containing hardener may also be applied to the hair. The hardener may be optional when silicon component has an amino active group and may be applied with the hydrophobic silicon containing constituent that contains active epoxy groups (Table-1) or applied in a composition consisting diglycidyl ether and reactive silicon composition. For example, a hardener may be applied to hair as a composition at an equivalent ratio to the constituent of the glycidyl ether (epoxy groups) for curing and bonding the hydrophobic silicon containing constituent to the hair. The hardener may comprise an amine terminated polypropyleneoxide, an amine terminated polybutadiene, isophorone diamine, meta xylene diamine or other hardener as is known by persons having ordinary skill in the art for hardening epoxy resins that consist the reactive hydrophobic silicon containing constituent or compound.

The hardener and the hydrophobic silicon containing constituent may be simultaneously applied to hair by mixing the hydrophobic silicon containing constituent with the hardener and/or epoxy reactive components and immediately applying to the hair. In at least one embodiment of the present disclosure, a reactive hydrophobic silicon containing constituent and a hardener are applied separately to the hair, in two separate applications. For example, a reactive hydrophobic silicon containing constituent may be sprayed, or otherwise applied, onto hair and separately, a hardener may be sprayed, or otherwise applied, onto the hair.

In at least one further embodiment, the hydrophobic silicon containing constituent, reactive constituent, and hardener are applied onto the hair simultaneously, with a sole application. For example, the hydrophobic silicon containing constituent, reactive constituent, and hardener, may all be mixed together and immediately applied onto the hair where it may cure in-situ.

The reactive constituent may have at least one functional group such as epoxy, amine, isocyanate, cyanoacrylate, or combinations thereof. An epoxy, amine, isocyanate, or cyanoacrylate functional group may be a part of a compound having the hydrophobic silicon containing constituent, or may be a separate constituent, which may be applied separately from, or with, the hydrophobic silicon containing constituent.

In at least one other illustrative example of the present disclosure, a method of mitigating dag formation on hair is provided. The method comprises applying a hydrophobic silicon containing constituent and a reactive constituent onto hair. The applying of the constituents enables a reaction of the reactive constituent with the hair and the transfer of the hydrophobicity of the hydrophobic silicon containing constituent to the hair. For example, the applications may enable a reaction of the reactive constituent with keratin or Cysteine in the hair and the holding of the hydrophobic silicon containing constituent to the hair. The hydrophobicity of the hydrophobic silicon containing constituent may then be transferred to the hair and thereby mitigate the formation of dag on the hair.

For example, one or more of the hydrophobic silicon containing constituents may comprise at least one amino group which may be curable with epoxy. Table 2, shows some examples of hydrophobic silicon containing constituents comprising at least one amino group that may be applied to hair to mitigate dag formation according to this disclosure.

TABLE 2

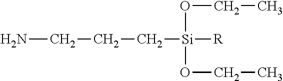

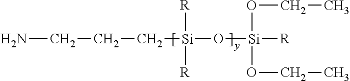

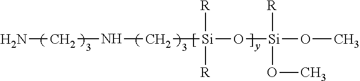

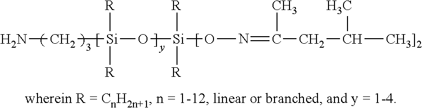

A reactive constituent comprising epoxy may be applied onto the hair at an equivalent ratio for curing and bonding the hydrophobic silicon constituent, which in the present illustrative example may comprise at least one amino group. The epoxy may be epoxy terminated polypropyleneoxide, diglycidylether of HTPB, epoxy terminated polyethyleneoxide, or butane diol diglycidylether.

In another illustrative example, the hydrophobic silicon containing constituent may comprise at least one cyanoacrylate group that may be polymerizable in-situ with the hair. Table 3, below, shows some examples of the hydrophobic siloxane containing constituents comprising at least one cyanoacrylate group that may be applied to hair to mitigate dag formation.

TABLE 3

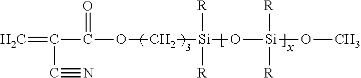

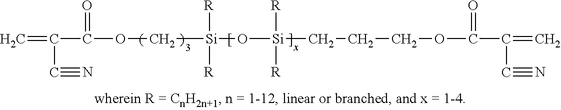

In a further illustrative example, the hydrophobic silicon containing constituent may comprise at least one isocyanate group and be polymerizable in-situ with the hair. For example, a hydrophobic siloxane containing constituent comprising at least one isocyanate group or in the presence of other polyurethane constituents (diisocyanate, diol, catalyst and cocatalyst) that may be applied to hair to mitigate dag formation may be represented with the structure of:

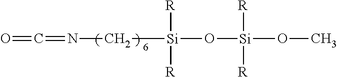

wherein R═CₙH₂ₙ₊₁, n=1-12, linear or branched.

The composition may have a catalyst to be applied onto the hair to accelerate curing reactions. The catalyst may be applied with the hydrophobic silicon containing constituent and/or the reactive constituent. The concentration of the catalyst required to control the rate of curing of the final silicon reactive coating is between 0.01-0.0001 weight % of the total composition.

The catalyst may comprise a composition selected from the group consisting of stanous octanoate, dialkyltin oxide, tetra-n-propyl orthosilicate, dioctyltin oxide, platinum complex catalysts, dibutyltin dilaurate, dibutyltin dioctanoate, and combinations thereof. The catalyst composition may have an inert solvent to dilute the catalyst as the concentration of the catalyst used is very low compared to the total composition.

The provided catalyst may be added to the container and mixed into the composition, the composition and provided catalyst may be simultaneously and immediately applied to the hair, immediately upon adding the provided catalyst to the container and mixing. Alternatively, the provided catalyst may be added to the solution, or applied to the hair, after the step of applying the composition onto the hair.

The composition may not have a catalyst where the atmospheric humidity will act as natural catalyst when the composition has cyanoacrylate, isocyanate, alkoxy, oxime, siloxane crosslinking agent.

In yet another illustrative example, the hydrophobic silicon containing constituent may comprise at least one siloxane cyclic monomer. For example, a hydrophobic silicon containing constituent comprising at least one siloxane cyclic tri siloxane monomer that may be applied to hair in the presence of some catalysts commonly known in the field of siloxane rubber and explained in previous embodiments to mitigate dag formation may be represented with the structure of:

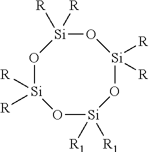

wherein R is hydrophobic alkyl group, CₙH₂ₙ₊₁, n=1-12, linear or branched, and R₁ is an active functional group, such as epoxy, amine, isocyanate, or cyanoacrylate.

The composition may have inert nontoxic solvents to be used to decrease the viscosity of the final composition to be spray able. Solvents suitable for such application may be acetone, other ketones, cyclohexane, diethylether or any other nontoxic volatile solvents, and combination thereof. The concentration of the solvent may vary between 10-80%.

The composition may consist reactive diluents that undergo polymerization during the curing of the siloxane active ingredients to be used to decrease the viscosity of the final composition to be spray able, reactive diluents suitable for such application may be cyclo alkene oxides, or alkyl glycidyl ether with chemical formula:

CₙH₂ₙ₊₁OCH₂CH(O)CH₂ (n=8-12), arylglycidylether C₆H₅OCH₂CH(O)CH₂, cyclohexenoxide, alkylarylglycidylethers, R—C₆H₄OCH₂CH(O)CH₂, or combination thereof.

The composition may consist a crosslinking agents selected from a group: RSi(OR⁻)₃R=alkyl (CₙH₂ₙ₊₁ n=1-18), C₆H₅, aryl, R—C₆H₄, siloxane, A crosslinking and binding active ingredient selected from the group consisting of CH₂(O)CHCH₂O—CH₂CH₂—CH₂Si(OC₂H₅)₃, NH₂CH₂CH₂CH₂Si(OC₂H₅)₃, C₆H₅—Si(—ON═C(CH₃)C₂H₅)₃, CH₃—Si(CH₃)₂—O(Si{CH₃}₂)ₙ—OCH₂CH(O)CH₂, and combinations thereof. R may have reactive functional groups such as amino, glycidyl, thiol, alkene, oxime and others.

The composition may consist reactive hydroxy or epoxy terminated polydimethyl siloxane. The hydroxy terminated polydimethyl siloxane may have different molecular weights, 750-150,000, with different viscosities (30-75000 cSt).

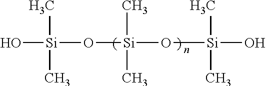

Hydroxy terminated Poly dimethyl siloxane

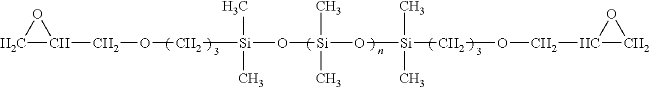

Epoxy terminated polydimethylsiloxane as reactive hydrophobic ingredient.

The composition may consist siloxane plasticiser comprise with formula: CH₃—Si(CH₃)₂—O(Si{CH3}₂)ₙ—O—Si(CH₃)₃, wherein n=3-5, in order to obtain flexible reactive siloxane hydrophobic coating.

The composition may consist internal siloxane plasticiser, hydrophobic siloxane structure and reactive functional group ingredients comprise for example but not limited with formula:

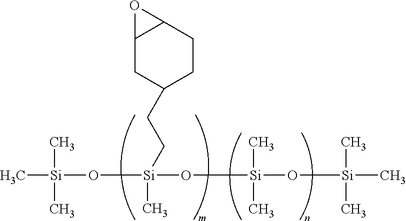

Preparation of the reactive hydrophobic siloxane based composition for dag prevention may be prepared by adding one or may be all the composition previously mentioned (hydroxyl terminated siloxane, a siloxane based plasticiser, a crosslinking and binding active ingredient, a solvent and a catalyst in stoichiometric quantities as detailed in the examples) into sealed spray able container filled under dry condition and sealed in the presence of spraying ingredient such as dimethyl ether or similar spraying aids or compressed dry nitrogen or any spraying technique commonly used with spraying materials.

Some of the hydrophobic siloxane containing constituents and/or reactive constituents may comprise monomers or active ingredients that may be prepared and some may be available commercially. Some reactive constituents may be examples listed in Table 4 may also be used as cross-linking agents for the hydrophobic silicon containing constituents of the present disclosure and for epoxy resins, and polyurethane.

TABLE 4

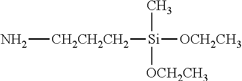

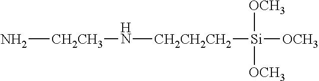

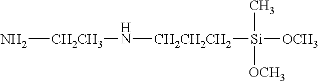

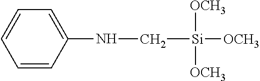 [SMILES: CO[Si](CNc1ccccc1)(OC)OC]

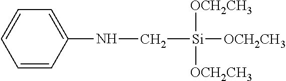 [SMILES: CCO[Si](CNc1ccccc1)(OCC)OCC]

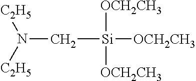 [SMILES: CCO[Si](CN(CC)CC)(OCC)OCC]

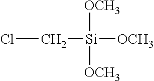

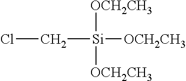 [SMILES: CCO[Si](CCl)(OCC)OCC]

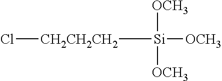

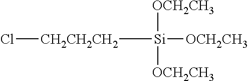

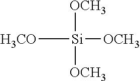

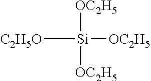

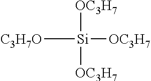

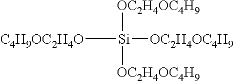

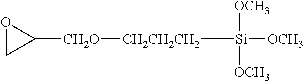

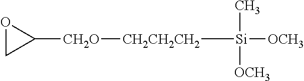

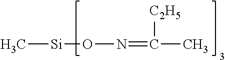

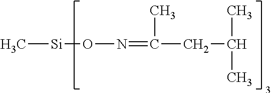

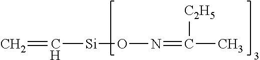

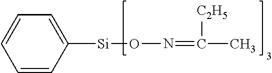

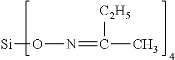

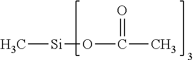

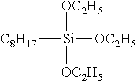

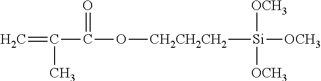

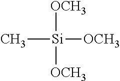

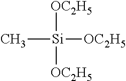

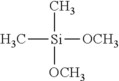

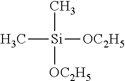

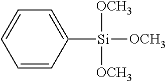 [SMILES: CO[Si](OC)(OC)c1ccccc1]

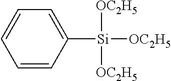 [SMILES: CCO[Si](OCC)(OCC)c1ccccc1]

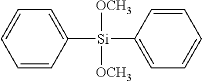 [SMILES: CO[Si](OC)(c1ccccc1)c1ccccc1]

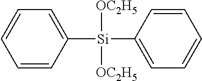 [SMILES: CCO[Si](OCC)(c1ccccc1)c1ccccc1]

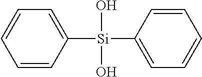 [SMILES: O[Si](O)(c1ccccc1)c1ccccc1]

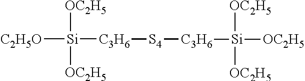

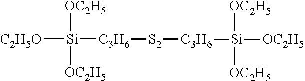

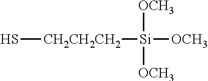

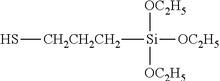

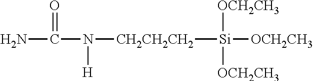

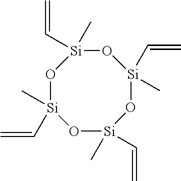 [SMILES: C=C[Si]1(C)O[Si](C)(C=C)O[Si](C)(C=C)O[Si](C)(C=C)O1]

Typical reactive siloxane active ingredients may be available commercially may be used in the composition of dag mitigation compositions of the present disclosure.

The steps of charging a container with: mono or di glycidyl terminated siloxane, and/or di or tri aliphatic or aromatic glycidylether such as, 1, 4-butane-dioldiglycidylether or 1, 1, 1-triglycidyltrimethylol propane, reactive diluent or a solvent such as cyclohexane to obtain suitable viscosity for spraying then adding a hardener such as meta xylene diamine in equivalent ratio as detailed in the examples. The obtained composition is mixed well and applied instantly to the hide. The step of curing the composition on the hair may comprise curing in ambient conditions for about 40 minutes.

The method may further include applying the composition detailed in paragraph [0065], without a hardener, the curing may be a photocatalytically. For example, curing may take place by subjecting the sprayed cattle to light, wherein the light enables activating a photocatalytic reaction and bonding the reactive hydrophobic siloxane composition with the hair. For example, a photo anionic catalyst or photo cationic catalyst, commonly used in photocopying ink, may be used.

There is thus provided a method for mitigating the formation of dag on hair.

In an illustrative method, a hydrophobic reactive silicon containing constituent a reactive constituent are applied to hair in the presence of inert solvent or reactive diluent. The hydrophobic reactive silicon containing constituent has at least one functional group selected from the group consisting of an epoxy group, a reactive amino group, a reactive cyanoacrylate group, a reactive isocyanate group, oxime, alkene, thiol and combinations thereof. The applied reactive constituent enables a reaction with the hair that binds the applied hydrophobic silicon to the hair and the applied hydrophobic reactive silicon containing constituent enables a transfer of its hydrophobicity to the hair to mitigate the formation of dag on the hair.

The hydrophobic reactive silicon containing constituent may be applied to the hair in the same application. The hydrophobic reactive silicon constituent may be reactive, have an epoxy group, and be selected from the group consisting of:

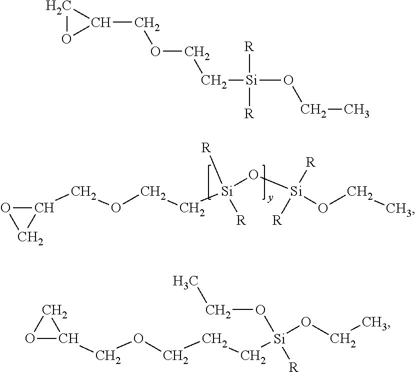

and combinations thereof; wherein R═CₙH₂ₙ₊₁, n=1-12, linear or branched, and y=1-4.

The method may further include applying a hardener onto the hair. The hardener and the reactive hydrophobic silicon may be applied at an equivalent ratio as detailed in the examples to cure and bond the reactive hydrophobic silicon containing constituent to the hair. The hardener may include an amine terminated polypropyleneoxide or an amine terminated polybutadiene, isophorone diamine, meta xylene diamine, hexamethylene diamine or other hardeners. The hydrophobic silicon containing constituent may comprise a reactive hydrophobic siloxane.

The reactive constituent may have at least one functional group selected from the group consisting of epoxy, amine, isocyanate, cyanoacrylate, alkoxy, thiol alkene and combinations thereof. The hydrophobic silicon containing constituent may include at least one amino group, is curable with epoxy, and is selected from the group consisting of:

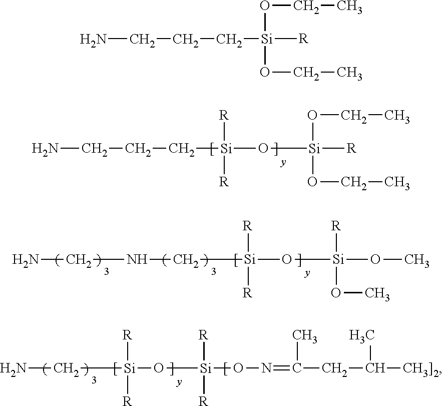

and combinations thereof; wherein R═CₙH₂ₙ₊₁, n=1-12, linear or branched, and y=1-4.

The reactive constituent may include an epoxy and the epoxy and the reactive hydrophobic silicon containing constituent may be applied at an equivalent ratio to cure and bond the hydrophobic silicon containing constituent to the hair. The epoxy may be epoxy terminated polydialkyl siloxane, epoxy terminated polypropylene oxide, epoxy terminated polybutadiene, epoxy terminated polyethyleneoxide, or butane diol diglycidylether.

The hydrophobic silicon containing constituent may include at least one cyanoacrylate group, is polymerizable in situ with the hair, and is selected from the group consisting of:

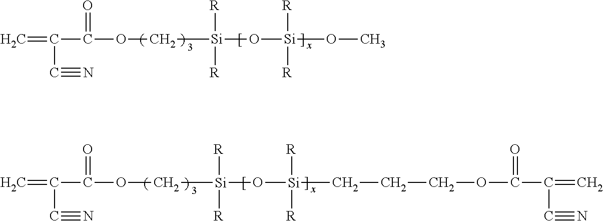

and combinations thereof.

The hydrophobic silicon containing constituent may include at least one isocyanate group, be polymerizable in situ with the hair, and may be represented with the structure of:

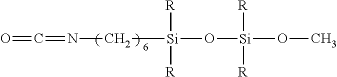

wherein R═CₙH₂ₙ₊₁, n=1-12, linear or branched; and applying a catalyst to the hair.

The hydrophobic silicon containing constituent may include at least one siloxane cyclic monomer represented with the structure of:

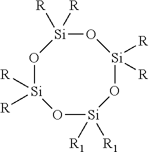

wherein R is hydrophobic alkyl group, CₙH₂ₙ₊₁, n=1-12, linear or branched, and may have an active functional group, R₁, selected from the group consisting of epoxy, amine, isocyanate, cyanoacrylate, alkene, thiol and combinations thereof.

In another illustrative embodiment, a method of mitigating dag formation on hair comprises: charging a container with hydroxy terminated siloxane; adding a siloxane plasticiser to the container; adding a crosslinking and binding active ingredient selected from the group consisting of CH₂(O)CHCH₂O—CH₂CH₂—CH₂Si(OC₂H₅)₃, NH₂CH₂CH₂CH₂Si(OC₂H₅)₃, C₆H₅—Si(—ON═C(CH₃)C₂H₅)₃, CH₃—Si(CH₃)₂—O(Si{CH₃}₂)ₙ—OCH₂CH(O)CH₂, and combinations thereof, to the container; adding an amount of solvent or reactive diluent to the container for obtaining a suitable viscosity of the composition, siloxane based plasticiser, and crosslinking and binding active ingredient, in the container, for applying to hair; mixing the hydroxy terminated siloxane, siloxane plasticiser, crosslinking and binding active ingredient, and solvent or diluent in the container to form a solution; applying the solution and a catalyst to the hair; wherein the catalyst enables a reaction of the applied solution with the hair that binds the applied hydroxy terminated siloxane to the hair and enables a transfer of its hydrophobicity to the hair to mitigate the formation of dag on the hair.

The catalyst and the solution may be pre-mixed before applying to the hair. The catalyst and the solution may be applied to the hair by separate applications. The hydroxy terminated siloxane may include a hydroxy terminated polydimethyl siloxane. The siloxane plasticiser may include CH₃—Si(CH₃)₂—O(Si{CH3}₂)ₙ—O—Si(CH₃)₃, wherein n=3-5, to the container. The solvent may be selected from the group consisting of cyclohexene oxide, cyclohexane, diethylether, acetone, and combinations thereof. The catalyst may be selected from the group consisting of stanous octanoate, dialkyltin oxide, tetra-n-propyl orthosilicate, dioctyltin oxide, platinum complex catalysts, dibutyltin dilaurate, dibutyltin dioctoate, and combinations thereof. The hydroxy terminated siloxane, the siloxane plasticiser, and the crosslinking and binding active ingredient may be added to the container at a ratio of about 42:3:8. The solution may include about 40% to about 60% solvent.

In another embodiment, a method of mitigating dag formation on hair may include the steps of: charging a container with hydroxy terminated siloxane; adding a siloxane plasticiser to the container; adding a crosslinking and binding active ingredient selected from the group consisting of CH₂(O)CHCH₂O—CH₂CH₂—CH₂Si(OC₂H₅)₃, NH₂CH₂CH₂CH₂Si(OC₂H₅)₃, C₆H₅—Si(—ON═C(CH₃)C₂H₅)₃, CH₃—Si(CH₃)₂—O(Si{CH₃}₂)ₙ—OCH₂CH(O)CH₂, and combinations thereof, to the container; adding an amount of solvent to the container for obtaining a suitable viscosity of the hydroxy terminated siloxane, siloxane plasticiser, and crosslinking and binding active ingredient, in the container, for spraying onto hair; mixing the hydroxy terminated siloxane, siloxane plasticiser, crosslinking and binding active ingredient, and solvent in the container to form a solution; spraying the solution onto the hair; and curing the solution on the hair, wherein the curing enables a reaction of the sprayed solution with the hair that binds the applied hydroxy terminated siloxane to the hair and enables a transfer of its hydrophobicity to the hair to mitigate the formation of dag on the hair. The curing may include a curing in ambient conditions for at least 15 minutes.

In another embodiment, a method of mitigating dag formation on hair may include the steps of: charging a container with glycidyl terminated siloxane; adding 1, 1, 1-triglycidyltrimethylol propane to the container; adding metaxylene diamine to the container; adding an amount of cyclohexane to the container for obtaining a suitable viscosity of the glycidyl terminated siloxane, 1, 1, 1-triglycidyltrimethylol propane, and metaxylene diamine, in the container, for spraying onto hair; mixing the cyclohexane, glycidyl terminated siloxane, 1, 1, 1-triglycidyltrimethylol propane, metaxylene diamine, and cyclohexane, in the container to form a solution; spraying the solution onto the hair; and curing the solution on the hair, wherein the curing enables a reaction of the sprayed solution with the hair that binds the applied glycidyl terminated siloxane to the hair and enables a transfer of its hydrophobicity to the hair to mitigate the formation of dag on the hair.

A diglycidyl terminated siloxane, illustratively shown below, and metaxylene diamine may be added to the container at an equivalent ratio. The solution may include about 0% to about 60% cyclohexane. The curing the solution on the hair may include curing in ambient conditions for about 40 minutes or curing activators may be added to accelerate curing.

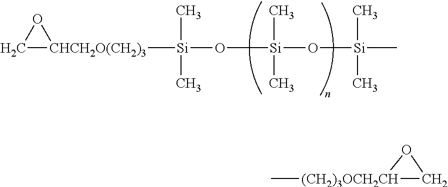

The diglycidyl terminated siloxane, and photocatalyst may be added to the container. The solution may include about 0% to about 60% solvent such as cyclohexane. The solution is sprayed to the hair and cured. Curing the solution on the hair may include curing in ambient conditions or photolytically, when the cattle is subjected to visible light.

In another illustrative embodiment, a method of mitigating dag formation on hair may include the steps of: charging a container with a first constituent selected from the group consisting of trialkyltrialkoxysilane such as: tri methoxy(2, 4, 4-trimethylpentyl) trialkoxysilane, or tri isobutyl tri alkoxy silane, and combinations thereof; adding a second constituent comprising alkyl trialkoxysilane that cosist at least one active functional group to enhance the bonding of the hydrophobic ingredient to the hair such as amino, epoxy, oxime, thiol, alken (Table-4) for example aminoethylaminopropyl triethoxy silane or 3-glycidyl ether propyl triethoxy silane, catalyst may be added to accelerate curing or may not be added to the container to form a solution; spraying the solution onto the hair; and curing the solution on the hair, wherein the curing enables a reaction of the sprayed solution with the hair to mitigate the formation of dag on the hair.

The following examples provide some illustrative experimental work of this disclosure.

### EXAMPLES

### Example 1

A 50 ml disposable container was charged with 10 g of tri methoxy(2, 4, 4-trimethylpentyl)silane, 2.5 g of aminoethylaminopropyl triethoxy silane, and 2.5 g of 1, 4-diglysidylether butane mixed well and then applied to the cattle hair, tissues paper, and glass slide or sprayed using small laboratory spraying set up, left to cure at ambient temperature, the hydrophobicity was tested after 24 hours, the contact angle of the surface was measured and found to be 135 showing good hydrophobic properties as shown in FIG. 2. The stability of the coated on the slide was tested by immersing the glass coated slide in water over night, the coat was found to be strongly bonded to the glass. The reaction takes place and polymerization of the reactive hydrophobic silicon ingredients will bond directly to any hydrophilic composition via strong epoxy bonding agent, such as keratin and melanin components, via the amino bonding group. The reaction may take place as illustrated in the reaction scheme shown in FIG. 4. However, it is to be understood that several types of reactions may take place at ambient temperature which are a part of the present method of mitigating dag.

### Example 2

A 50 ml spraying bottle was charged with 10 g of trimethoxy isobutyl silane, 2.5 g of 3-glycidyl ether propyl triethoxy silane and 2.5 g of aminoethylaminopropyl triethoxy silane mixed well then sprayed to cattle Hyde, tissue paper, and glass slide. Then left to cure at ambient temperature, the hydrophobicity was tested after 24 hours, the contact angle of the surface was measured and found to be 121 showing good hydrophobic properties as shown in FIG. 2. The stability of the coated glass slide was tested by immersing the glass coated slide in water over night, the coat was found to be strongly bonded to the glass. The reaction takes place and polymerization of the reactive hydrophobic silicon ingredients will bond directly to any hydrophilic composition via strong epoxy bonding agent, such as keratin and melanin components, via the amino-epoxy curing forming bonding group. The reaction may take place as illustrated in the reaction scheme shown in FIG. 5. However, it is to be understood that several types of reactions may take place at ambient temperature which are a part of the present method of mitigating dag.

### Example 3

A 50 ml disposable container was charged with 8.4 g of hydroxyl terminated polydimethylsiloxane viscosity 35 cSt., 1.47 g of 3-glycidyl ether propyl triethoxy silane, and 0.6 g of methyl terminated polydimethylsiloxane plasticizer, and 0.001 g of the stannous octanoate catalyst, mixed well and then applied to the cattle hair, tissues paper, and glass slide and sprayed using small laboratory spraying set up, left to cure at ambient temperature, the hydrophobicity was tested after 24 hours, the contact angle of the surface was measured and found to be 139 showing good hydrophobic properties as shown in FIG. 2. The stability of the coated on the slide was tested by immersing the glass coated slide in water over night, the coat was found to be strongly bonded to the glass. The reaction takes place and polymerization of the reactive hydrophobic silicon ingredients will bond directly to any hydrophilic composition via strong epoxy bonding agent, such as keratin and melanin components, via the amino bonding group. The reaction may take place according to the well-known curing reactions of silicone rubber. However, it is to be understood that several types of reactions may take place at ambient temperature which are a part of the present method of mitigating dag. The reaction takes place and polymerization of the reactive hydrophobic silicon ingredients bonded directly to any hydrophilic composition of the keratin components, via the glycidyl ether reaction with amino groups and alkoxy reactive groups linked to silicon.

Experimental procedures, curing and testing methods explained in examples 1-3 were adopted in the following examples using different silane reactive starting material, crosslinking agents, and reactive bonding ingredients.

### Example 4

A container was charged with 14 g of hydroxyl terminated polydimethyl siloxane with viscosity 100 cSt., 1 g siloxane based plasticizer, 9 g of cyclohexane solvent, 4 g of phenyl tri (ethylmethylketoxime)silane and 0.0003 g of stannous octanoate catalyst. The obtained reactive bonded coat has contact angle equal to 135, with excellent hydrophobicity and bonding stability.

### Example 5

A container was charged with 8.4 g of hydroxyl terminated polydimethyl siloxane with viscosity 100 cSt, 0.6 g siloxane based plasticizer, 8 g of cyclohexaneoxide as reactive diluent, and 1.47 g aminoethylaminopropyltrimethylsilane. The obtained reactive bonded coat has contact angle equal to 121, with very good hydrophobicity and bonding stability.

### Example 6

A container was charged with 28 g of diglycidyl ether terminated polydimethylsiloxane with molecular weight 80000, 2.68 g phenyl tri(ethylmethylketoxime), 2.5 g aminoethylaminopropyltrimethoxysilane, and 7.5 g cyclohexane mixed well and applied, the measured contact angle was 128 with very good hydrophobicity.

### Example 7

A container was charged with, 14 g of hydroxyterminated polydimethylsiloxane (viscosity 75 cSt), 1 g plasticizer, 2.5 g aminoethylaminopropyl trimethoxy silane, 1 g of 1, 1, 1-triglycidyltrimethylol propane, 0.4 g metaxylene diamine, 10 g cyclohexane, 0.0003 g stanous octanoate catalyst. The homogenous solution was mixed well and applied in the container forming a solution onto the hair, tissue paper and glass slide and left to cure at ambient temperature. The applied and sprayed product cured within two hours and left overnight, the hydrophobic coat was tested, the contact angle was 114, with good hydrophobicity.

### Example 8

A container was charged with 14 g of 3, 4, 4-trimethyl pentyl trimethoxy silane and 2.5 g of aminoethylaminopropyltrimethoxy silane as a reactive bonding agent, the mixture was applied and sprayed, the product cured with catalytic humidity within two hours and left overnight, the hydrophobic coat was tested, the contact angle was 122, with good hydrophobicity.

### Example 9

A container is charged with 10 g of diisocyanate terminated polydimethylsiloxane, 1 g of hexamethylene diisocyanate and 1 g of polyether polyol, 0.005 g cobalt octanoate catalyst, the composition is applied to the tissue paper, hide and glass slide, left to cure then to be tested. Good bonding and good hydrophobicity is exhibited.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of mitigating dag on hair, the method comprising:
applying a hydrophobic silicon containing constituent and a reactive constituent to hair;
wherein the hydrophobic silicon containing constituent has at least one reactive functional group selected from the group consisting of an epoxy group, a reactive amino group, a reactive cyanoacrylate group, a reactive isocyanate group, oxime, thiol, alkene and combinations thereof; and
wherein the reactive constituent enables a reaction with the hair that binds the hydrophobic silicon containing constituent to the hair and the applied hydrophobic silicon containing constituent imparts its hydrophobicity to the hair to mitigate the formation of dag on the hair.

2. The method of claim 1, wherein the hydrophobic silicon containing constituent and the reactive constituent are applied to the hair together.

3. The method of claim 1, wherein the hydrophobic silicon containing constituent and the reactive constituent are applied to the hair separately.

4. The method of claim 1, wherein the hydrophobic silicon containing constituent is reactive, has an epoxy group, and is selected from the group consisting of: 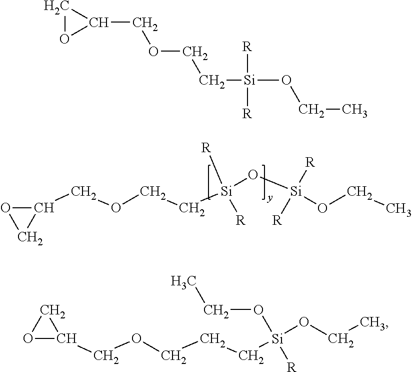
and combinations thereof;
wherein R═CₙH₂ₙ₊₁, n=1-12, linear or branched, and y=1-4.

5. The method of claim 4, further comprising applying a reactive epoxy or alkene group that can polymerize photocatalytically in the presence of a photocatalyst onto the hair and exposing the hair to light, wherein the light enables activating a photocatalytic reaction and the bonding of the reactive hydrophobic silicon containing constituent with the hair.

6. The method of claim 4 further comprising applying a hardener onto the hair and curing.

7. The method of claim 6, wherein the hardener comprises an epoxy and the reactive hydrophobic silicon comprises one or more epoxy reactive groups and are applied at an equivalent ratio to cure and bond the hydrophobic silicon containing constituent to the hair.

8. The method of claim 6, wherein the hardener comprises an amine terminated polypropyleneoxide, an amine terminated polybutadiene, isophorone diamine, triethylene tetra amine, tetraethylenepenta amine, their aducts, aminosiloxane active ingredient crosslinking agent, or aminoethylaminopropyltrimothoxy silane.

9. The method of claim 1, wherein the hydrophobic silicon containing constituent comprises at least one of polydialkyl siloxane, hydroxyterminated polydialkyl siloxane, methyl terminated polydialkyl siloxane, isocyanate terminated polydialkyl siloxane, cyanoacrylate terminated polydialkyl siloxane, and alkene terminated polydialkyl siloxane.

10. The method of claim 1, wherein the reactive constituent has at least one functional group selected from the group consisting of epoxy, amine, isocyanate, cyanoacrylate, thiol, alkene, oxime and combinations thereof.

11. The method of claim 1, wherein the hydrophobic silicon containing constituent comprises at least one amino group, is curable with epoxy, and is selected from the group consisting of: 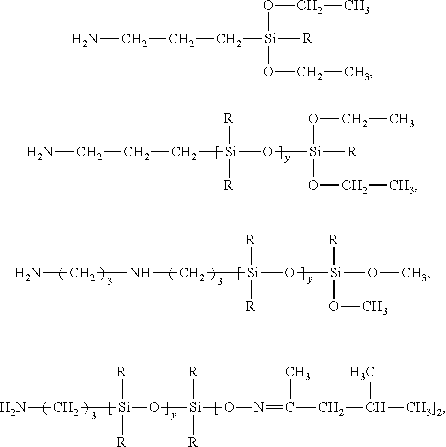
and combinations thereof;
wherein R═CₙH₂ₙ₊₁, n=1-12, linear or branched, and y=1-4.

12. The method of claim 11, wherein the reactive constituent comprises an epoxy or a reactive constituent having amino groups and the hydrophobic silicon constituent and the reactive constituent are applied at an equivalent ratio to cure and bond the hydrophobic silicon containing constituent to the hair.

13. The method of claim 12, wherein the epoxy is epoxy terminated polypropyleneoxide, epoxy terminated polybutadiene, epoxy terminated polyethyleneoxide, butane diol diglycidylether, or epoxy terminated polydimethylsiloxane.

14. The method of claim 1, wherein the hydrophobic silicon containing constituent comprises siloxane and at least one cyanoacrylate group, is polymerizable in situ with the hair, and is selected from the group consisting of: 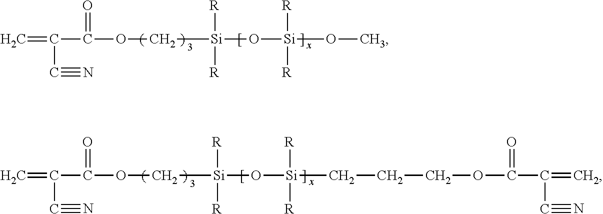
and combinations thereof.

15. The method of claim 1, wherein the hydrophobic silicon containing constituent comprises at least one isocyanate group, is polymerizable in situ with the hair, and is represented with the structure of: 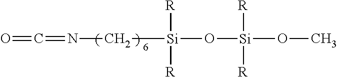
wherein R═CₙH₂ₙ₊₁, n=1-12, linear or branched.

16. The method of claim 1, wherein the hydrophobic silicon containing constituent comprises at least one siloxane cyclic monomer represented with the structure of: 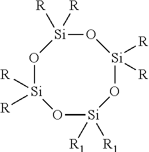
wherein R is hydrophobic alkyl group, CₙH₂ₙ₊₁, n=1-12, linear or branched, and R₁ is an active functional group selected from the group consisting of epoxy, amine, isocyanate, cyanoacrylate, and combinations thereof.

17. A method of mitigating dag formation on hair, the method comprising:
charging a container with hydroxy terminated siloxane;
adding a siloxane plasticiser to the container;
adding a crosslinking and binding active ingredient selected from the group consisting of CH₂(O)CHCH₂O—CH₂CH₂—CH₂Si(OC₂H₅)₃, NH₂CH₂CH₂CH₂Si(OC₂H₅)₃, C₆H₅—Si, CH₃—Si(CH₃)₂—O(Si{CH₃}₂)ₙ, OCH₂CH(O)CH₂, and combinations thereof, to the container;
adding an amount of solvent or reactive diluent to the container for obtaining a suitable viscosity of a composition in the container;
adding a catalyst in range of about 0.001-0.0001 weight % of the composition, in the container, for applying to hair;
mixing the composition in the container to form a homogenous solution;
applying the solution to the hair; and
wherein the catalyst enables or accelerates a reaction of the applied solution with the hair that binds the applied hydroxy terminated siloxane to the hair and enables a transfer of its hydrophobicity to the hair to mitigate dag on the hair.

18. The method of claim 17, wherein the hydroxy terminated siloxane comprises a hydroxy terminated polydialkylsiloxane, polydiphenylsiloxane, poly(dialkylpenylene) siloxane, poly(alkyl phenyl)siloxane, or polydimethyl siloxane.

19. The method of claim 17, wherein the siloxane plasticiser comprises alkyl terminated polydialkylsiloxane or CH₃—Si(CH₃)₂—O(Si{CH3}₂)ₙ—O—Si(CH₃)₃, wherein n=3-5.

20. The method of claim 17, wherein the solvent or reactive diluent is selected from the group consisting of cyclohexane, diethylether, acetone, and combinations thereof.

21. The method of claim 17, wherein the solvent or reactive diluent is selected from the group consisting of cyclo alkene oxides, alkyl glycidyl ether with chemical formula of CₙH₂ₙ₊₁OCH₂CH(O)CH₂ (n=8-12), arylglycidylether, C₆H₅OCH₂CH(O)CH₂, cyclohexenoxide, alkylarylglycidylethers, R—C₆H₄OCH₂CH(O)CH₂, and combination thereof.

22. The method of claim 17, wherein the catalyst is selected from the group consisting of stanous octanoate, dialkyltin oxide, tetra-n-propyl orthosilicate, dioctyltin oxide, platinum complex catalysts, dibutyltin dilaurate, dibutyltin dioctoate, and combinations thereof.

23. The method of claim 17, wherein the hydroxy terminated siloxane, the siloxane plasticiser, and the crosslinking and binding active ingredient are added to the container at a ratio between about 10:1:2 and 10:0:2.

24. The method of claim 17, wherein the solution comprises up to about 0-60% solvent for obtaining a sprayable or applicable composition.

25. A method of mitigating dag formation on hair comprising the steps of:
charging a container with hydroxy terminated siloxane;
adding a siloxane plasticiser to the container;
adding a crosslinking and binding active ingredient selected from the group consisting of CH₂(O)CHCH₂O—CH₂CH₂—CH₂Si(OC₂H₅)₃, NH₂CH₂CH₂CH₂Si(OC₂H₅)₃, C₆H₅—Si([—ON═C(CH₃)C₂H₅]₃], CH₃—Si(CH₃)₂—O(Si{CH₃}₂)ₙ—OCH₂CH(O)CH₂, and combinations thereof, to the container;
adding an amount of solvent to the container for obtaining a suitable viscosity of the hydroxy terminated siloxane, siloxane plasticiser, and crosslinking and binding active ingredient, in the container, for spraying onto hair;
mixing the hydroxy terminated siloxane, siloxane plasticiser, crosslinking and binding active ingredient, and solvent in the container to form a solution;
spraying the solution onto the hair; and
curing the solution on the hair, wherein the curing enables a reaction of the sprayed solution with the hair that binds the applied hydroxy terminated siloxane to the hair and enables a transfer of its hydrophobicity to the hair to mitigate dag on the hair.

26. The method of claim 26, wherein the curing comprises curing in ambient conditions for at least 15 minutes.

27. A method of mitigating dag formation on hair, the method comprising:
charging a container with glycidyl terminated polydialkyl siloxane;
adding diglycidylether or triglycidylether to the container;
adding an epoxy curing hardener to the container;
adding an amount of solvent or active diluent to the container for obtaining a suitable viscosity of the compositions in the container, for spraying or applying onto hair;
mixing the container to form a solution;
spraying or applying the solution onto the hair; and
curing the solution on the hair, wherein the curing enables a reaction of the sprayed solution with the hair that binds the applied glycidyl terminated siloxane to the hair and enables a transfer of its hydrophobicity to the hair to mitigate dag on the hair.

28. The method of claim 27, wherein the glycidyl terminated siloxane, and the diglycidylether or triglycidylether, and the hardener are added to the container at an equivalent ratio for curing.

29. The method of claim 27, wherein the solution comprises about 40% to about 60% solvent or reactive diluent.

30. The method of claim 27, wherein the curing the solution on the hair comprises curing in ambient conditions for about 40 minutes.

31. A method of mitigating dag formation on hair comprising the steps of:
charging a container with a first constituent selected from the group consisting of trialkoxyalkylsilane, tri methoxy(2, 4, 4-trimethylpentyl)silane, triethoxy isobutyl silane, and combinations thereof;
adding a second constituent comprising high bonding active crosslinking agent selected from the group consisting of aminoethylaminopropyl triethoxy silane, 3-glycidyl ether propyl triethoxy silane, and combinations thereof, to the container;
adding a third constituent comprising a transition metal alkanoate catalyst for acceleration of curing;
wherein the first constituent, second constituent, and third constituent are charged or added to the container at a weight ratio between about 10:1:0.001 to 4:1:0.001;
mixing the first constituent, second constituent, and third constituent in the container to form a solution;
spraying the solution onto the hair; and
curing the solution on the hair, wherein the curing enables a reaction of the sprayed solution with the hair to mitigate dag on the hair.
